(19) United States
(12) Patent Application Publication
(10) Pub. No.: US 2021/0219994 A1
(43) Pub. Date: Jul. 22, 2021

(54) BURR WITH IRRIGATION AND IMAGING

(71) Applicant: Wright Medical Technology, Inc., Memphis, TN (US)

(72) Inventors: Erin MULLER, Lakeland, TN (US); Paul LUTTRELL, Germantown, TN (US); Brian Robert THOREN, Memphis, TN (US); Dean J. NACHTRAB, Memphis, TN (US); Joseph Ryan WOODARD, Memphis, TN (US)

(73) Assignee: Wright Medical Technology, Inc., Memphis, TN (US)

(21) Appl. No.: 16/980,060

(22) PCT Filed: Jun. 27, 2018
(86) PCT No.: PCT/US2018/039664

§ 371 Date: Sep. 11, 2020

(51) Int. Cl. A61B 17/16; A61B 17/02; A61B 1/04; A61B 1/07; A61B 1/00

(52) U.S. Cl. CPC A61B 17/1644; A61B 17/0218; A61B 1/04; A61B 2017/00296; A61B 1/00066; A61B 1/00009; A61B 2217/007; A61B 1/07

(57) ABSTRACT

A tool includes a burr having a first shaft configured for rotation or oscillation. The burr has a plurality of cutting edges at a first end of the burr. The first shaft has a central longitudinal passage extending from the first end to a second end of the first shaft. A camera is mounted adjacent the first end of the first shaft.

### FIELD

This disclosure is related to surgical tools.

### BACKGROUND

Mid-foot Charcot procedures are performed to return a patient's foot to plantar grade. Incision length and procedure time are interest for Charcot procedures. Many Charcot procedures use open surgical treatment techniques. A surgeon performs a “wedge cut” and bone may be removed, or bone and soft tissues may be left in place if of good bone quality. A wedge cut provides an acute angular resection across the foot that enables the surgeon to remove “bad” bone and then close the two resection ends of the bone back together. Because the cuts were made at an acute angle, when the bone is drawn back together, the acute angle creates an arch in the foot, eliminating the flatfoot/cavus condition.

Tools suitable for Charcot procedures and/or minimally invasive surgical procedures are desired.

### SUMMARY

In some embodiments, a tool comprises a burr having a first shaft configured for rotation or oscillation. The burr has a plurality of cutting edges at a first end thereof. The first shaft has a central longitudinal passage extending from the first end to a second end of the first shaft. A camera is mounted adjacent the first end of the first shaft.

In some embodiments, a tool comprises a camera. A guide has a first passage with an inner wall. The inner wall defines: a second passage for conducting light therethrough, a third passage for the camera or a camera coupling, and a fourth passage for conducting a fluid therethrough. A burr has a rotatable shaft with a plurality of cutting edges at a first end thereof. The rotatable shaft is insertable through the first passage so that the first end of the shaft extends from a first end of the guide. The camera is connected to output an image or video signal via the camera coupling.

In some embodiments, a method comprises collecting image data in a wound site using a camera on a shaft, while the camera is positioned adjacent a first end of a burr, and the shaft extends through the burr. Bone is removed using the burr. A fluid is delivered through the burr, while removing the bone.

In some embodiments, a method comprises collecting image or video data in a wound site using a camera on an end of a burr. The burr is rotated or reciprocated so as to remove material from a bone, while collecting the image data. The image or video data is processed using image stabilization.

### BRIEF DESCRIPTION OF THE DRAWINGS

FIGS. 1A and 1B are isometric drawings of an exemplary tool for cutting and/or grinding bone.

FIG. 2 is an enlarged detail showing the cannulated burr of FIG. 1.

FIGS. 3A and 3B show an embodiment of a burr having a detachable camera.

FIG. 4 shows a burr having fenestrations.

FIG. 5 shows a guide catheter with channels for fluid and light.

FIG. 6 shows a variation of the guide catheter of FIG. 5.

FIG. 7 shows a variation of the guide catheter of FIG. 5 or FIG. 6 having a grommet at the distal end.

FIGS. 8A to 8C show a conical guide catheter with channels for fluid and light.

FIGS. 8D and 8E show a conical guide catheter with a handle.

FIGS. 9A to 9C show variations of the conical guide catheter.

FIG. 10 is a flow chart of a method of using the burr of FIG. 1.

### DETAILED DESCRIPTION

This description of the exemplary embodiments is intended to be read in connection with the accompanying drawings, which are to be considered part of the entire written description. In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

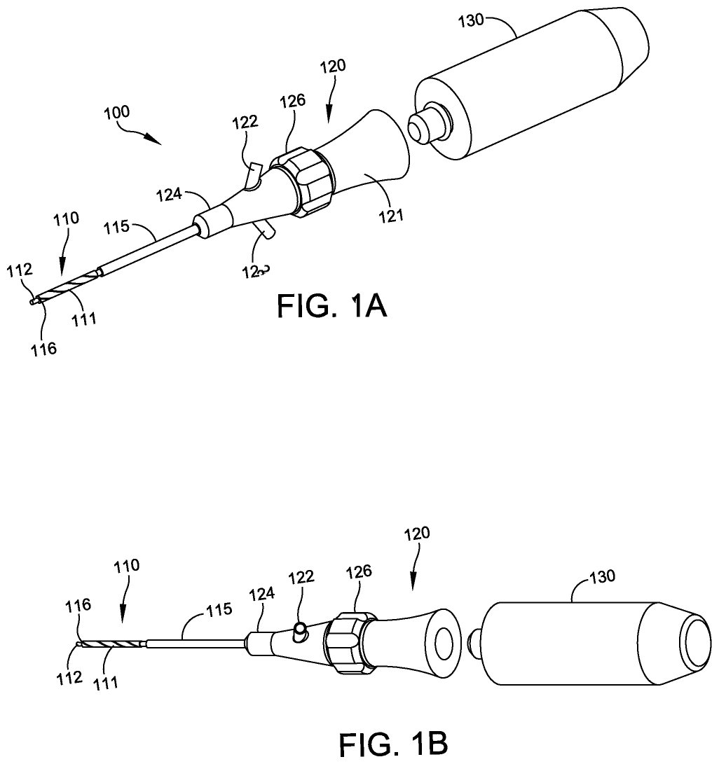

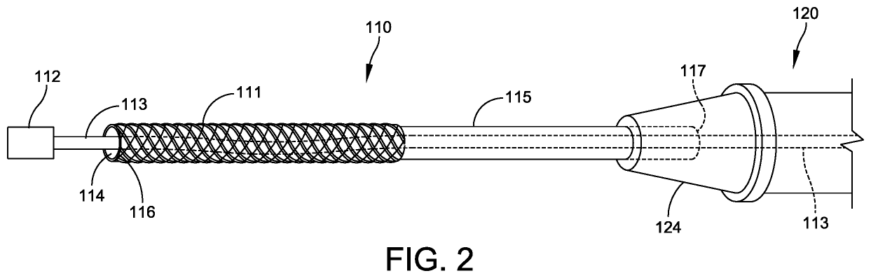

FIGS. 1A, 1B and 2 show a grinding tool 100 comprising a burr 110. In some embodiments, the burr 110 has a first shaft 115 configured for rotation or oscillation. The burr can be used for grinding, reaming, or cutting. The burr 110 has a plurality of cutting or grinding edges 111 at a first (distal) end 116 thereof. As best seen in FIG. 2, the burr 110 can be cannulated. The first shaft 115 and the distal end 116 having the cutting or grinding edges 111 can both have a central longitudinal passage 114 extending through the first shaft 115, from the first (distal) end 116 to the second (proximal) end 117. Although the first (distal) end of the burr 110 has a cylindrical cutting or grinding edge 111, in other embodiments (not shown in FIGS. 1A-2), the distal end 116 of burr 110 can have another shape, such as, but not limited to a sphere, a hemisphere, an ellipsoid, a cone, a paraboloid, a frustum, or a rounded capsule (i.e., a cylinder having a hemispherical end). The cutting or grinding edge 111 can have a helical thread, or a single cut, double cut, diamond cut, or knurled edge, for example.

The burr 110 can couple with a micro-camera 112 to provide intra-articular or intraosseous visibility, e.g., for minimally invasive surgery. In some embodiments, a camera 112 is mounted at the distal end of a second shaft 113, adjacent the first (distal) end 116 of the first shaft 115. The camera 112 can have a complementary metal oxide semiconductor (CMOS) sensor or a charge-coupled device (CCD) sensor. The camera 112 can have a size in a direction transverse to the longitudinal axis from about 1 mm to about 1.5 mm. For example the camera 112 can have a diameter of about 1.2 mm. In some embodiments, the camera 112 has a wide field of view (FOV). For example, the camera 112 can have an FOV of at least 64 degrees. In some embodiments, the camera 112 can have an FOV of at least 84 degrees. In some embodiments, the camera 112 can have an FOV in a range from 100 degrees to 130 degrees. In some embodiments, the camera 112 can be a micro-camera, such as the “MICRO SCOUTCAM™” 1.2 camera with an associated “MICRO SCOUTCAM™” digital signal processor (DSP) video processor”, both sold by Medigus, Ltd. of Omer, Israel. The camera 112 can have a wired connection (not shown) or wireless connection to the DSP video processor. The DSP video processor can be coupled via wired or wireless connection to a display (not shown), for viewing by the surgeon.

The camera 112 can be mounted on a second shaft 113 (FIG. 2). The second shaft 113 is rotatably positionable in the central longitudinal passage 114 of the first shaft 115. The outer diameter of the second shaft 113 is smaller than the inner diameter of the first shaft 115, defining an annular central longitudinal passage 114 between the second shaft 113 and the first shaft 115. In some embodiments, the tool 100 is configured for delivering a fluid through the central longitudinal passage 114 to the first (distal) end 116 of the first shaft 115. For example, a saline solution can be provided via the central longitudinal passage 114 to prevent excessive heating of the burr 110 and/or the bone being cut or ground, and to flush out bone fragments and/or soft tissue from the wound site to prevent buildup. In some embodiments, the second shaft 113 is flexible. In other embodiments, the second shaft 113 is rigid. In some embodiments, the burr 110 and the second shaft 113 comprise stainless steel or titanium.

In some embodiments, the tool 100 has a handle 120 configured for gripping the second (camera) shaft 113, and rotating the first (burr) shaft 115. The handle 120 has a stationary portion 121 on which at least one port is provided. The handle 120 of FIGS. 1A and 1B has two ports 122 and 123, for light and saline, respectively.

The light port 122 can be connected by an optical fiber (not shown in FIGS. 1A-2) to the distal end of the burr 110 for illuminating the tissue within the wound site. In some embodiments, the optical fiber (not shown) can extend longitudinally along the length of the second shaft 113 to a point at or near the distal end of the second shaft 113, providing light near the camera 112 and directing the light into the FOV of the camera 112.

The saline port 123 can be connected to a pump (not shown). In some embodiments, the saline port 123 is used to provide saline (or water or other sterilized fluid) to flush out any fragments of bone, cartilage and/or soft tissue in the wound site. In some embodiments, the saline port 123 can be connected to a vacuum (e.g., a pump operated in a reverse direction) to remove small fragments and particles from the wound site. In some embodiments, as shown in FIG. 2, the central longitudinal passage 114 of the burr 110 delivers all of the saline (or other fluid) to the distal end 116 of the burr 110, near the camera 112.

The handle 120 has a motor (not shown) and motorized chuck 124 for holding and rotating the first shaft 115 of the burr 110. A knob 126 or set screw (not shown) can be provided to fixedly grip the second shaft 113, so the burr 110 can rotate for removing material while the second shaft 113 holds the camera stationary (relative to the handle 120). In some embodiments, the shaft 115 is slidably or threadably held within the handle 120 and can be extended or retraced longitudinally, independently of the burr 110. For example, the camera 112 can be deployed in the position shown in FIGS. 1A and 1B for examining the tissue to be cut or ground, and then withdrawn inside the shaft 115 to protect the camera 112 while grinding a bone. The handle 120 can be connected to a power source 130 which can contain a battery (not shown) or provide an alternating current connection for power.

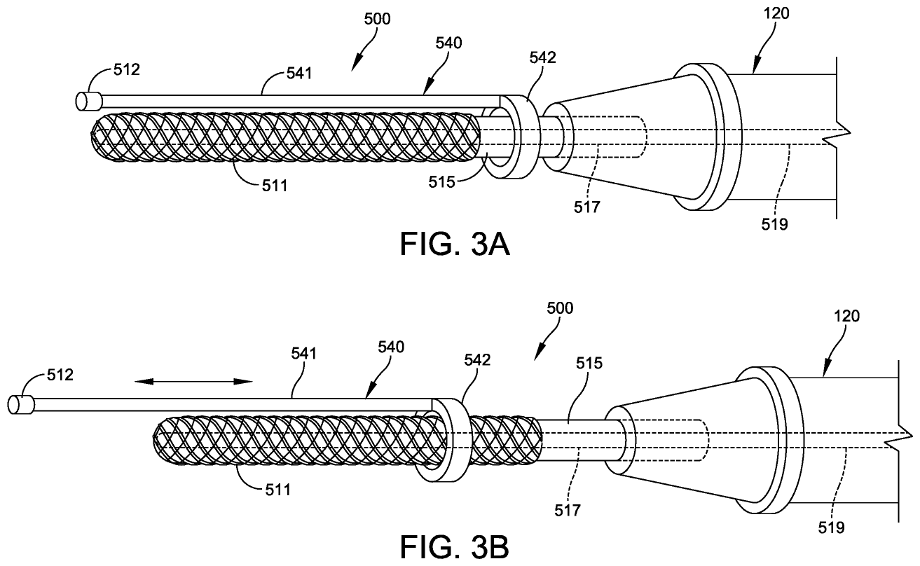

FIGS. 3A and 3B show an alternative embodiment of the tool comprising a burr 500 with a cutting or grinding surface 511. A micro-camera 512 is mounted on an arm 541 extending longitudinally from a collar 542. The collar 542 is configured to rotate with the burr 500 (or cutting instrument).

The arm 541 and collar 542 can be a unitary (single-piece) mounting device 540 for attaching the camera 512 to the burr 500. In other embodiments (not shown), the arm 541 can be a separate member joined to the collar 542 (e.g., by mating threads). In some embodiments, the collar 542 can fit tightly around the shaft 515 of the burr 500. In other embodiments, the collar 542 has a set screw (not shown) for fixing the collar 542 to the shaft 515.

As shown in FIG. 3B, the mounting device 540 can be removed from the burr 500, sterilized, and reused with a different burr (not shown). In some embodiments, either the collar 542 or the shaft 515 has one or more detents (not shown) to allow the collar 542 to shift between two or more positions and click into place. Although FIGS. 3A and 3B show the collar 542 fixed to the shaft 515, in other embodiments (not shown), the collar is fixed to the handle 120 and does not rotate with the burr, reducing motion blur and simplifying image stabilization.

Because the camera 512 is fixed to the shaft 515, both burr 540 and camera 512 rotate during cutting or grinding of the bone. In some embodiments, the camera 512 is used while the burr 511 is not rotating, to obtain clear images or video. In other embodiments, if the camera 512 has a fast exposure time (e.g., 30 frames/second) and the burr 511 is being used for slow-speed reaming (e.g., 330 RPM or less), the camera 512 can be used while the drill is rotating.

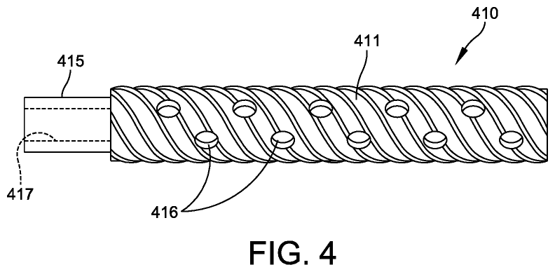

FIG. 4 shows another embodiment of a burr 410 having a shaft 415 and an outer circumferential surface 411 with cutting edges. The burr 410 is cannulated, and has a central longitudinal passage 417. The burr 410 has a plurality of radial fenestrations 416 extending radially from the central longitudinal passage 417 to the outer circumferential surface 411. The burr 410 can provide saline, water, or the like at multiple locations along the side of burr 410 to cool the bone while the burr 410 grinds the bone.

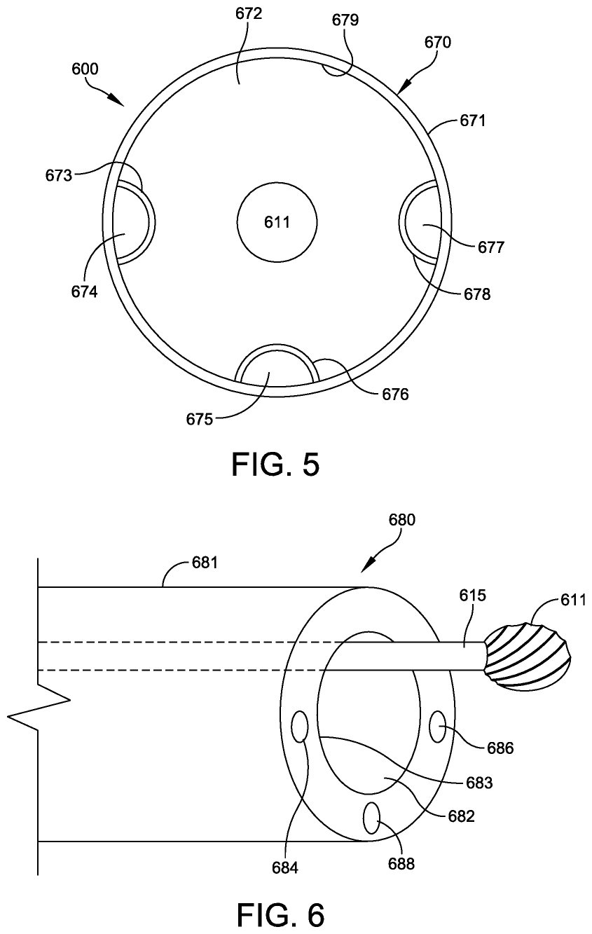

FIG. 5 shows an embodiment of a multi-component catheter system 600. The catheter 670 provides a guide having a first passage 672 with an outer wall 671 and an inner wall 679. A second passage wall 673 and a portion of the inner wall 679 define a second passage 674 for conducting light therethrough. A third passage wall 678 and a portion of the inner wall 679 define a third passage 677 for the camera 112 (not shown in FIG. 6) and/or a camera coupling. The camera coupling can be a wired or wireless connection to a DSP video processor (not shown in FIG. 6). In some embodiments, the camera coupling can include an optical fiber. In some embodiments, the camera 112 can extend from the third passage 677. For example, the camera 112 can be mounted on a shaft 113 of a type shown in FIG. 2. In other embodiments, the camera 112 can be flush with or recessed within the third passage 677. In some embodiments, a fourth passage wall 676 and another portion of the inner wall 679 defines a fourth passage 675 for conducting a fluid (e.g., saline or water) therethrough. The proximal end of the fourth passage 675 can be coupled to the saline port 123 (FIG. 1A). The cannula 670, second passage wall 673, third passage wall 678, and fourth passage wall 676 may be extruded.

In some embodiments, as shown in FIG. 5, the catheter 670 provides light and saline, and a camera mounting, and a burr 611 passes through the first passage 672. The burr 611 may be cannulated or non-cannulated. The burr 611 may not have any light or saline source.

The catheter 670 has with two or more channels 674, 675, 677 for providing light, providing water, and receiving an optical or electrical signal from a camera (not shown). A central passage 672 can receive a burr 611 or cutting component. In some embodiments, tubes 673, 676 and 678 defining channels 674, 675, and 677 are extruded on the inside wall of the catheter. Although FIG. 5 shows three channels 674, 675, and 677, other embodiments can have two, four or more channels for additional functions, such as and power, vacuum, ultraviolet (UV) light, etc.

FIG. 6 shows a variation of the catheter 680, having an outer wall 681, an inner wall 683, and longitudinal passages 684, 686, and 688. The catheter 680a serves as a guide having a first passage 682 with an outer wall 681 and an inner wall 683.

The inner wall 683 defines a first passage, through which a burr 611 or cutting tool is extended on a shaft 615. The longitudinal passages 684, 686 and 688 may be formed between the outer wall 681 and the inner wall 682. In some embodiments, the catheter 680 and passages 684, 686, 688 can be formed by extrusion or by additive manufacturing.

In some embodiments, as shown in FIG. 6, the light channel 684 and water channel 686 can be molded into the sidewall 681 of the catheter 680 and a shaft 615 with the burr 611 mounted on the distal end thereof can pass through the center of the catheter. In some embodiments, a camera 688 can be recessed in the end surface at the distal end of the catheter. By recessing the camera 688 within the wall of the catheter, the camera can be protected while advancing the catheter 680 into the wound site. Although FIG. 6 shows three channels 684, 686, and 688, other embodiments can have two, four or more channels for additional functions, such as vacuum, ultraviolet (UV) light, etc.

A burr 611 is mounted on a shaft 615. The shaft 615 can be a rotatable shaft, having a smooth side surface or a plurality of cutting edges at a first (distal) end of the shaft 615. The rotatable shaft 615 is insertable through the first passage 682 so that the first (distal) end of the shaft 615 extends from a first end of the guide 682 as shown. A camera is connected to output an image or video signal via the camera coupling. In some embodiments, the camera 688 is flush with or recessed in the distal end of the catheter 680.

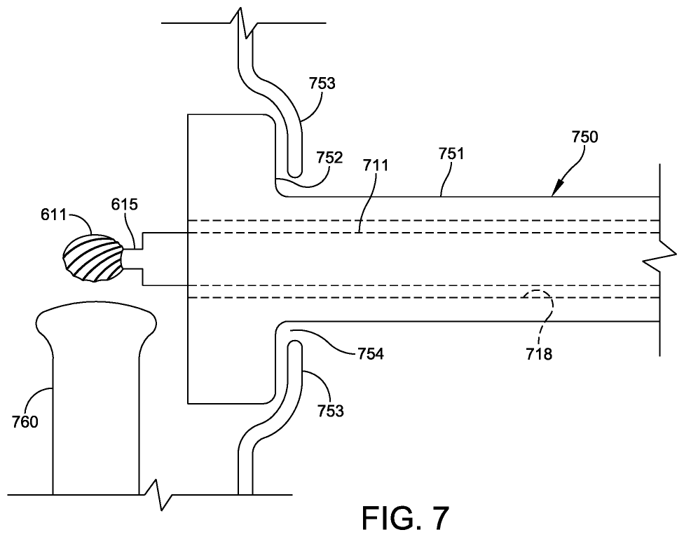

FIG. 7 shows a variation of the catheter, comprising a guide 750 with a grommet 752 at the first (distal) end of the guide 750. The grommet 752 is adapted to be inserted within an incision 754 in a skin 753 of a patient to protect the skin 753 around the guide 750. The guide 750 has an inner wall 718 with a hollow cylindrical shape defining a working channel.

The grommet style end 752 provides stability of the working channel 718 and protects the skin 753 around the working channel 718. The grommet 752 can be worked through the small incision site 754 and sits underneath the skin. The cylinder 851 is the only portion of the guide 750 protruding from the skin 753 and provides the access/working channel 718.

FIG. 7 shows the guide 750 in situ, with a burr 611 extending from the guide 750. The burr 611 can be a non-cannulated burr mounted on a shaft 615 as described in the discussion of FIG. 6. In other embodiments (not shown), the burr can be a cannulated burr 110 as described above with respect to FIG. 2, including a camera 112 on a shaft 115 extending from the passage 114 of the burr 110, and can have cutting or grinding edges 111. The burr 110 is positioned to grind a bone 760.

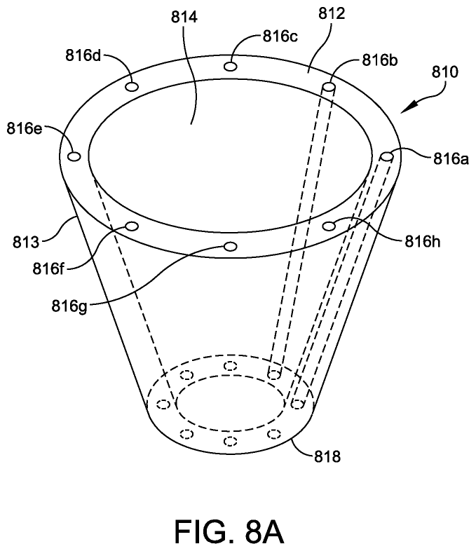

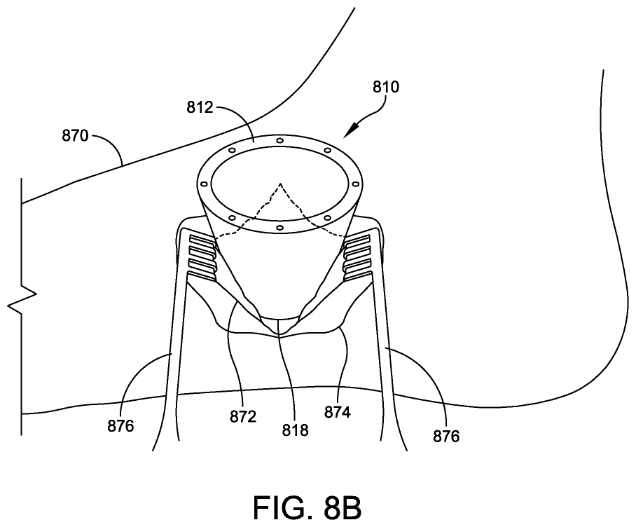

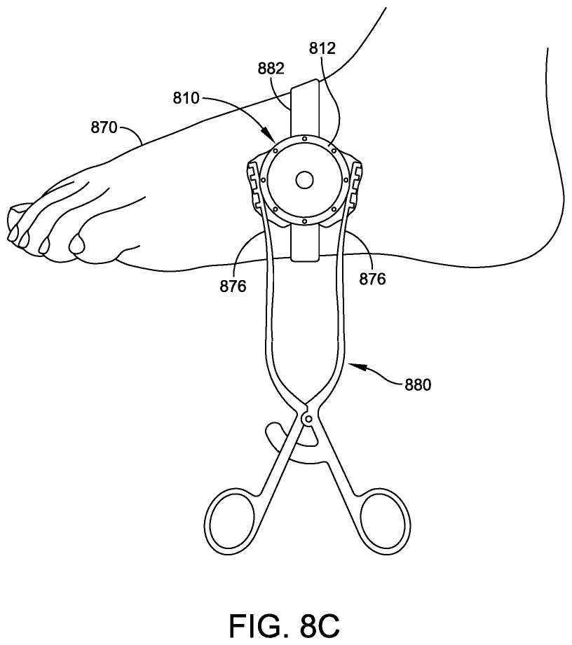

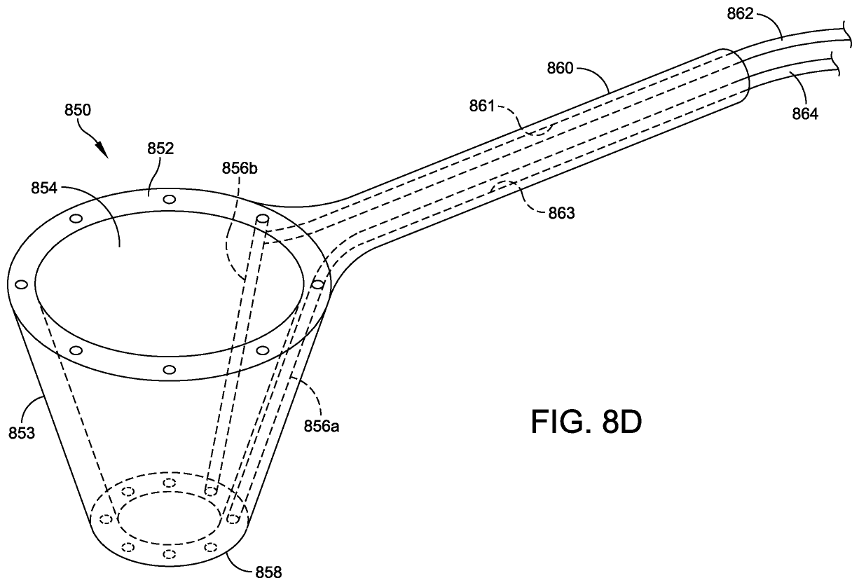

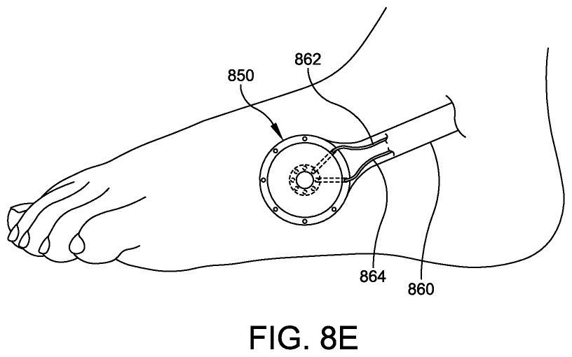

FIG. 8A shows an embodiment of a multi-component catheter system 810. The catheter 670 provides a guide having a first passage 672 with an outer wall 671 and an inner wall 679. A second passage wall 673 and a portion of the inner wall 679 define a second passage 674 for conducting light therethrough. A third passage wall 678 and a portion of the inner wall 679 define a third passage 677 for the camera 112 (not shown in FIG. 6) and/or a camera coupling. The camera coupling can be a wired or wireless connection to a DSP video processor (not shown

FIGS. 8A-8C show another embodiment in which the guide 810 has a conical frustum shape. The conical frustum shape allows for greater range of motion of those items being inserted into the wound space. The shape allows the surgeon to vary the angle of the burr 611 during grinding or cutting, and may allow the surgeon to access bone surfaces which cannot be reached with a perpendicular burr 611. The guide 810 has an outer frustum-shaped surface 813 and an inner frustum shaped surface 814, a top annular edge 812, and a bottom annular edge 818.

The bottom annular edge 818 of the guide 810 is open to allow for insertion and removal of elements, such as instrumentation, tissue, fluid, or the like. These elements can be inserted and/or removed through the opening in the bottom annular edge 818.

A plurality of channels 816a-816h extend through the wall between the outer surface 813 and the inner surface 814. The channels extend from the top annular edge 812 to the bottom annular edge 818. The plurality of channels 816a-816h allow for greater visibility and access to the surgical site 754 (FIG. 7). Lights, fluids, instruments, tissue, heat pipes, cautery systems, etc. can be inserted and/or removed through these channels 816a-816h. Although FIG. 8A shows eight channels 816a-816h, any number of channels can be provided. In the example, the channels 816a and 816b provide paths for supplying saline and light, respectively.

The system can further include at least one spreader 876 attached to the first end of the guide 810. FIG. 8B shows two spreaders 876 on opposite sides of the guide 810. Once an incision is made, the spreaders 876 part the skin 874 to expose the wound site 872. The guide 810 can then be inserted.

FIG. 8C shows the inserted guide 810 with the spreaders 876 of the retractor 880 still in place. A strap 882 can be attached to the bottom annular edge 818 at the first (distal) end of the guide 810 to hold the guide 810 in place. With the guide 810 strapped in place, the burr 611 (FIG. 6) can be used to grind or cut a bone.

FIGS. 8D and 8E show a variation of the guide 850 with a handle 860 for positioning and holding the guide 850 in place. The handle 860 can have a plurality of channels 861, 863 therethrough, for conducting fluid, light or the like to or from the guide 860. Each channel 861, 863 is connected to a respective channel 856a, 856b of the guide 860 extending through the handle 860. Although FIGS. 8D and 8E show a handle 860 with two channels 861, 863, any desired number of channels can be included. The channels 861, 863 can be connected to respective feeds 862, 864 for fluid, light, power or the like. FIG. 8E shows the guide 850 held in place by the handle 860 during the cutting or grinding procedure. As discussed above for the conical guide 810 of FIGS. 8A-8C, the guide 850 has separate channels for light, fluid(s), instruments, tissue, heat pipes, cautery systems, etc.

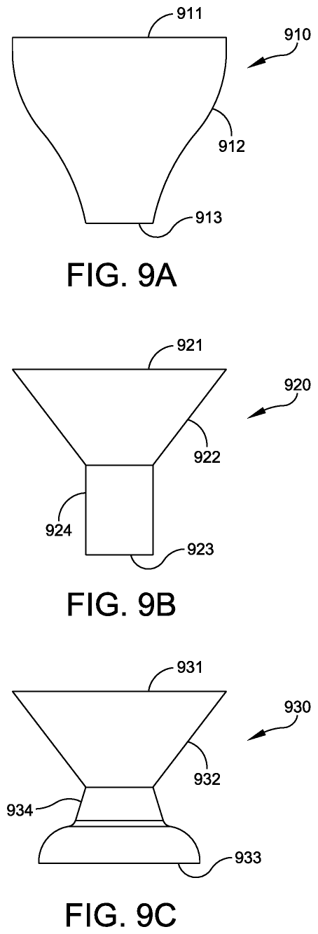

Although FIGS. 8A-8E show guides 810, 850 having a conical frustum shape, the guides can have other shapes. For example, FIG. 9A shows a guide 910 having a smoothly curved outer surface 912 with an inflection point 914. Above the inflection point 914, the surface 912 is convex in cross section. Below the inflection point, the surface 912 is concave in cross-section. The inner surface (not shown) of the guide 910 can be curved concentrically with outer surface 912 (for a constant wall thickness). In other embodiments, the inner surface (not shown) of the guide 910 can be a conical frustum (for a varying wall thickness). Other embodiments of the guide can have other smoothly curved surface shapes.

FIG. 9B shows a funnel-shaped guide 920 having a conical frustum portion 922 beginning at the top end 921 and a cylindrical portion 924 ending at the bottom end 923. In some embodiments, the inner surface (not shown) of the guide 920 has a frustum portion at the top end and a cylindrical portion ending at the bottom end, concentrically located with surfaces 922 and 924 for a constant wall thickness. In other embodiments, the inner surface (not shown) of the guide 910 can be a conical frustum (for a varying wall thickness).

FIG. 9C shows another variation of a guide 930 having a conical frustum portion 932 at the top end 931 and a grommet 933 at the bottom end. A connecting portion 934 connects the conical frustum portion 932 to the grommet 933. The connecting portion can be an inverted conical frustum (as shown), smoothly curved, or cylindrical. The grommet 933 can protect the skin the same way as the grommet 752 discussed above with respect to FIG. 7.

The shapes in FIGS. 8A-9C are only exemplary, and the guide can have other variations in shape. Any of the guides 910, 920 or 930 can be held in place by a strap (as shown in FIG. 8C or a handle as shown in FIGS. 8D and 8E. For example, instead of small cylindrical channels as shown in FIGS. 8A-8E, the channels can be arc shaped and can subtend angles from about 20 degrees to about 85 degrees, providing a wider flow path for fluid or light.

All of the burrs and cutting tools described herein can comprise hard materials, such as stainless steel, tungsten carbide, polycrystalline diamond, combinations thereof, or the like. In some embodiments, the burrs and cutting tools can have a coating, such as black oxide, titanium nitride, titanium aluminum nitride, titanium carbon nitride, diamond, zirconium nitride.

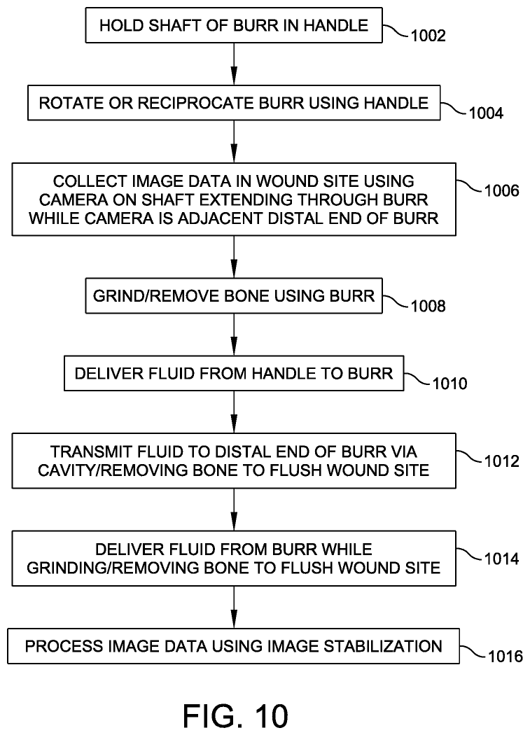

FIG. 10 is a flow chart of an exemplary method of cutting or grinding a bone.

At step 1002, the surgeon holds the shaft of the burr 110 in a handle 120.

At step 1004, the surgeon rotates or reciprocates the burr 110. The handle 120 causes the rotating or reciprocating.

At step 1006, the camera 112 collects image data in a wound site. The camera 112 is positioned on a shaft 115, while the camera 112 is positioned adjacent a first end 117 of a burr 110, and the shaft 115 extends through the burr 110.

At step 1008, the surgeon grinds or removes bone material using the burr 110.

At step 1010, fluid (e.g., saline, water) is provided from the handle 120 to the burr 110.

At step 1012, The fluid is transmitted through the burr, to the distal end 116 of the burr 110 via a cavity 114 between an inner wall of the burr 110 and the shaft 115. while removing the bone.

At step 1014, the fluid is delivered from the burr 110 while grinding or removing bone, to flush the wound site. In some embodiments (FIG. 1), the fluid is delivered from the distal end of the bur 110. In some embodiments (FIG. 4), the burr 410 has a plurality of fenestrations 416 on a side thereof, and the step of delivering the fluid includes injecting the fluid radially through the fenestrations 416.

At step 1016, the processor for the camera processes the image data using image stabilization.

Although the subject matter has been described in terms of exemplary embodiments, it is not limited thereto. Rather, the appended claims should be construed broadly, to include other variants and embodiments, which may be made by those skilled in the art.

What is claimed is:

1. A tool comprising:
a burr having a first shaft configured for rotation or oscillation, the burr having a plurality of cutting edges at a first end thereof, the first shaft having a central longitudinal passage extending from the first end to a second end of the first shaft; and
a camera that is mounted adjacent the first end of the first shaft.

2. The tool of claim 1, wherein the camera is mounted on a second shaft, and the second shaft is rotatably positionable in the central longitudinal passage of the first shaft.

3. The tool of claim 2, further comprising a handle configured for:
gripping the second shaft,
rotating the first shaft, and
delivering a fluid through the central longitudinal passage to the first end of the first shaft.

4. The tool of claim 1, wherein the first shaft has a plurality of radial fenestrations extending from central longitudinal passage to an outer circumferential surface of the first shaft.

5. A tool comprising:
a camera;
a guide having a first passage with an inner wall, the inner wall defining:
a second passage for conducting light therethrough, and
a third passage for the camera or a camera coupling,
a fourth passage for conducting a fluid therethrough;
a burr having a rotatable shaft with a plurality of cutting edges at a first end thereof, the rotatable shaft being insertable through the first passage so that the first end of the shaft extends from a first end of the guide;
and
wherein the camera is connected to output an image or video signal via the camera coupling.

6. The tool of claim 5, further comprising a camera holder including a collar adapted to grip the shaft at a second end of the shaft opposite the first end, and an arm configured to extend from the collar to the first end of the shaft, the camera mounted on an end of the arm opposite the collar.

7. The tool of claim 5, further comprising a handle configured for:
gripping the guide,
rotating the shaft inside the guide, and
delivering the fluid through the third passage to the first end of the shaft.

8. The tool of claim 5, wherein the guide has a grommet at the first end thereof, the grommet adapted to be inserted within an incision in a skin of a patient.

9. The tool of claim 5, wherein the guide has a hollow cylindrical shape.

10. The tool of claim 5, wherein the guide has a conical frustum shape.

11. The tool of claim 5, further comprising at least one spreader attached to the first end of the guide.

12. The tool of claim 5, further comprising at least one strap attached to the first end of the guide.

13. A method comprising:
collecting image data in a wound site using a camera on a shaft, while the camera is positioned adjacent a first end of a burr, and the shaft extends through the burr;
removing bone using the burr; and
delivering a fluid through the burr, while removing the bone.

14. The method of claim 13, further comprising;
holding the shaft in a handle,
rotating the burr, wherein the handle causes the rotating.

15. The method of claim 14, further comprising;
providing the fluid from the handle to the burr.

16. The method of claim 13, wherein the fluid is delivered to the first end of the burr via a cavity between an inner wall of the burr and the shaft.

17. The method of claim 13, wherein the burr has a plurality of fenestrations on a side thereof, and delivering the fluid includes injecting the fluid radially through the fenestrations.

18. A method comprising:
collecting image or video data in a wound site using a camera on an end of a burr;
rotating or causing the burr to reciprocate so as to remove material from a bone, while collecting the image data; and
processing the image or video data using image stabilization.
